(19) United States
(12) Patent Application Publication
(10) Pub. No.: US 2020/0368409 A1
(43) Pub. Date: Nov. 26, 2020

(54) FLEXIBLE, DISPOSABLE FLUID COLLECTION CONTAINER FOR NEGATIVE-PRESSURE THERAPY

(71) Applicant: KCI Licensing, Inc., San Antonio, TX (US)

(72) Inventor: Christopher Brian LOCKE, Bournemouth (GB)

(73) Assignees: KCI Licensing, Inc., San Antonio, TX (US); KCI Licensing, Inc., San Antonio, TX (US)

(21) Appl. No.: 16/966,326

(22) PCT Filed: Feb. 11, 2019
(86) PCT No.: PCT/US2019/017494

§ 371 Date: Jul. 30, 2020

Related U.S. Application Data
(60) Provisional application No. 62/630,544, filed on Feb. 14, 2018.

(51) Int. Cl. A61M 1/00; A61F 13/00

(52) U.S. Cl. CPC A61M 1/0088; A61M 1/0017; A61F 13/00068; A61M 2205/502; A61M 2205/3331; A61M 2205/18; A61M 2205/52; A61M 2205/7536

(57) ABSTRACT

In one example embodiment, a system for treating a tissue site with reduced pressure may include a distribution component, a negative-pressure source, and a collection vessel. The collection vessel may include a rigid canister defining a chamber. The collection vessel may also include a flexible container. The flexible container may be configured to receive fluid from the distribution component and to provide pressure communication while restricting liquid communication between an internal volume of the flexible container and the chamber of the rigid canister when the flexible container is disposed within the chamber of the rigid canister. The flexible container may include a port configured to provide the pressure communication and to restrict liquid communication between the internal volume of the flexible container and the chamber of the rigid canister. The port may include a hydrophobic filter.

### RELATED APPLICATIONS

The present invention claims the benefit, under 35 USC § 119(e), of the filing of U.S. Provisional Patent Application Ser. No. 62/630,544, entitled “Flexible, Disposable Fluid Collection Container For Negative-Pressure Therapy,” filed Feb. 14, 2018. This provisional application is incorporated herein by reference for all purposes.

### TECHNICAL FIELD

The subject matter set forth in the appended claims relates generally to tissue treatment systems and more particularly, but without limitation, to apparatuses, systems, and methods for the treatment of a tissue site with negative pressure.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for a therapy including the provision of negative pressure are set forth in the appended claims. Illustrative embodiments are also provided to enable a person skilled in the art to make and use the claimed subject matter.

For example, in some embodiments a system for treating a tissue site with reduced pressure may comprise a distribution component, a negative-pressure source, and a fluid-collection vessel. The fluid-collection vessel may comprise a rigid canister defining a chamber. The fluid-collection vessel may also comprise a flexible container. The flexible container may be configured to receive a fluid from the distribution component and to provide a route of pressure communication while restricting liquid communication between an internal volume of the flexible container and the chamber of the rigid canister when the flexible container is disposed within the chamber of the rigid canister. The flexible container may comprise a first port configured to provide the route of pressure communication between the internal volume of the flexible container and the chamber of the rigid canister. The first port may be configured to restrict liquid communication between the internal volume of the flexible container and the chamber of the rigid canister. The first port may comprise a hydrophobic filter. The hydrophobic filter may be configured to allow pressure communication and to restrict liquid communication. The flexible container may also comprise a second port configured to provide the route of pressure communication between the internal volume of the flexible container and the chamber of the rigid canister. The first port may be disposed on a first surface of the flexible container and the second port may be disposed on a second surface of the flexible container.

Also for example, in some embodiments is a vessel for collecting liquid from a distribution component adapted to be fluidly coupled to a tissue site for treatment with a reduced pressure treatment. The vessel may comprise a rigid canister defining a chamber. The vessel may also comprise a flexible container. The flexible container may be configured to receive a fluid from the distribution component and to provide a route of pressure communication while restricting liquid communication between an internal volume of the flexible container and the chamber of the rigid canister when the flexible container is disposed within the chamber of the rigid canister. The flexible container may comprise a first port configured to provide the route of pressure communication between the internal volume of the flexible container and the chamber of the rigid canister. The first port may be configured to restrict liquid communication between the internal volume of the flexible container and the chamber of the rigid canister. The first port may comprise a hydrophobic filter. The hydrophobic filter may be configured to allow pressure communication and to restrict liquid communication. The flexible container may also comprise a second port configured to provide the route of pressure communication between the internal volume of the flexible container and the chamber of the rigid canister. The first port may be disposed on a first surface of the flexible container and the second port may be disposed on a second surface of the flexible container.

Also for example, in some embodiments is a method for treating a tissue site with reduced pressure. The method may comprise sealing the tissue site at a distribution component, fluidly coupling a negative-pressure source to the distribution component, drawing fluid from the distribution component with the negative-pressure source, and collecting at least a portion of the fluid in a vessel. The vessel may comprise a rigid canister defining a chamber. The vessel may also comprise a flexible container. The flexible container may be configured to receive a fluid from the distribution component and to provide a route of pressure communication while restricting liquid communication between an internal volume of the flexible container and the chamber of the rigid canister when the flexible container is disposed within the chamber of the rigid canister. The flexible container may comprise a first port configured to provide the route of pressure communication between the internal volume of the flexible container and the chamber of the rigid canister. The first port may be configured to restrict liquid communication between the internal volume of the flexible container and the chamber of the rigid canister. The first port may comprise a hydrophobic filter. The hydrophobic filter may be configured to allow pressure communication and to restrict liquid communication. The flexible container may also comprise a second port configured to provide the route of pressure communication between the internal volume of the flexible container and the chamber of the rigid canister. The first port may be disposed on a first surface of the flexible container and the second port may be disposed on a second surface of the flexible container.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an embodiment of a therapy system for use in providing negative pressure therapy in accordance with this specification;

FIG. 2 is a schematic view of an embodiment a vessel for use in the therapy system of FIG. 1;

FIG. 3 is a simplified illustration of a flexible container for use with the vessel of FIG. 2;

FIG. 4 is a simplified cross-sectional view of a canister for use with the vessel of FIG. 2; and

FIG. 5 is a schematic view of an additional embodiment a vessel for use in the therapy system of FIG. 1.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

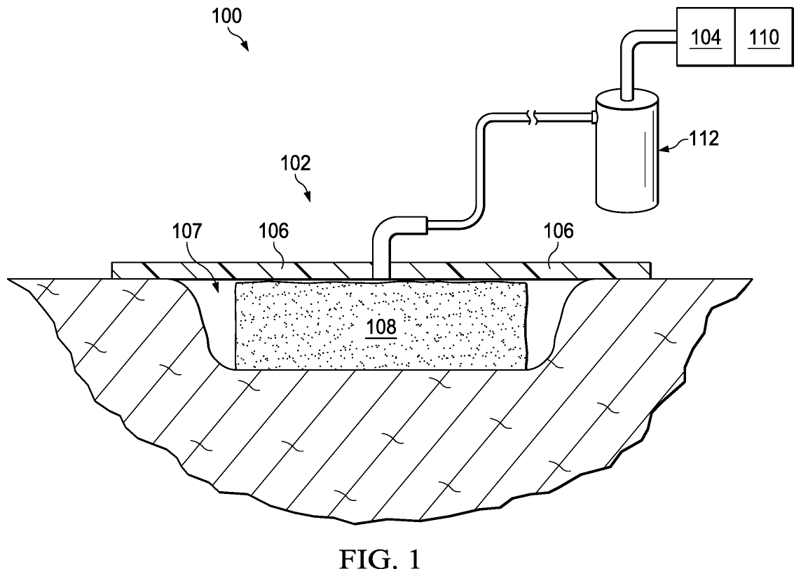

FIG. 1 is a simplified functional block diagram of an example embodiment of a therapy system 100 that can provide negative-pressure therapy in accordance with this specification.

The term “tissue site” in this context broadly refers to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include negative-pressure supply, and may include or be configured to be coupled to a distribution component, such as a dressing. In general, a distribution component may refer to any complementary or ancillary component configured to be fluidly coupled to a negative-pressure supply in a fluid path between a negative-pressure supply and a tissue site. A distribution component is preferably detachable, and may be disposable, reusable, or recyclable. For example, a dressing 102 may be fluidly coupled to a negative-pressure source 104, as illustrated in FIG. 1. A dressing may include a cover, a tissue interface, or both in some embodiments. The dressing 102, for example, may include a cover 106 and a tissue interface 108. A regulator or a controller, such as a controller 110, may also be coupled to the negative-pressure source 104.

In some embodiments, a dressing interface may facilitate coupling the negative-pressure source 104 to the dressing 102. For example, such a dressing interface may be the SENSA T.R.A.C.™ Dressing available from Acelity L.P. of San Antonio, Tex.

Additionally, the therapy system 100 may include sensors to measure operating parameters and provide feedback signals to the controller 110 indicative of the operating parameters. As illustrated in FIG. 1, for example, the therapy system 100 may include a pressure sensor 120, an electric sensor 122, or both, coupled to the controller 110. The pressure sensor 120 may also be coupled or configured to be coupled to a distribution component and to the negative-pressure source 104.

Components may be fluidly coupled to each other to provide a path for transferring fluids (i.e., liquid and/or gas) between the components. For example, components may be fluidly coupled through a fluid conductor, such as a tube. A “tube,” as used herein, broadly includes a tube, pipe, hose, conduit, or other structure with one or more lumina adapted to convey a fluid between two ends. Typically, a tube is an elongated, cylindrical structure with some flexibility, but the geometry and rigidity may vary. In some embodiments, components may also be coupled by virtue of physical proximity, being integral to a single structure, or being formed from the same piece of material. Moreover, some fluid conductors may be molded into or otherwise integrally combined with other components. Coupling may also include mechanical, thermal, electrical, or chemical coupling (such as a chemical bond) in some contexts. In general, components of the therapy system 100 may be coupled directly or indirectly. For example, the negative-pressure source 104 may be directly coupled to the controller 110.

The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of a fluid “inlet” or “outlet” in such a frame of reference. This orientation is generally presumed for purposes of describing various features and components herein. However, the fluid path may also be reversed in some applications (such as by substituting a positive-pressure source for a negative-pressure source) and this descriptive convention should not be construed as a limiting convention.

“Negative pressure” generally refers to a pressure less than a local ambient pressure, such as the ambient pressure in a local environment external to a sealed therapeutic environment provided by the dressing 102. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. Similarly, references to increases in negative pressure typically refer to a decrease in absolute pressure, while decreases in negative pressure typically refer to an increase in absolute pressure. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

A negative-pressure supply, such as the negative-pressure source 104, may be a reservoir of air at a negative pressure, or may be a manual or electrically-powered device that can reduce the pressure in a sealed volume, such as a vacuum pump, a suction pump, a wall suction port available at many healthcare facilities, or a micro-pump, for example. A negative-pressure supply may be housed within or used in conjunction with other components, such as sensors, processing units, alarm indicators, memory, databases, software, display devices, or user interfaces that further facilitate therapy. For example, in some embodiments, the negative-pressure source 104 may be combined with the controller 110 and other components into a therapy unit. A negative-pressure supply may also have one or more supply ports configured to facilitate coupling and de-coupling the negative-pressure supply to one or more distribution components.

The tissue interface 108 can be generally adapted to contact a tissue site. The tissue interface 108 may be partially or fully in contact with the tissue site. If the tissue site is a wound, for example, the tissue interface 108 may partially or completely fill the wound, or may be placed over the wound. The tissue interface 108 may take many forms, and may have many sizes, shapes, or thicknesses depending on a variety of factors, such as the type of treatment being implemented or the nature and size of a tissue site. For example, the size and shape of the tissue interface 108 may be adapted to the contours of deep and irregular shaped tissue sites. Moreover, any or all of the surfaces of the tissue interface 108 may have projections or an uneven, course, or jagged profile that can induce strains and stresses on a tissue site, which can promote granulation at the tissue site.

In some embodiments, the tissue interface 108 may be a manifold. A “manifold” in this context generally includes any substance or structure providing a plurality of pathways adapted to collect or distribute fluid across a tissue site under pressure. For example, a manifold may be adapted to receive negative pressure from a source and distribute negative pressure through multiple apertures across a tissue site, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, the pathways of a manifold may be interconnected to improve distribution or collection of fluids across a tissue site. In some illustrative embodiments, a manifold may be a porous foam material having interconnected cells or pores. For example, cellular foam, open-cell foam such as a reticulated foam, porous tissue collections, and other porous material such as gauze or felted mat generally include pores, edges, and/or walls adapted to form interconnected fluid channels. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

The average pore size of a foam may vary according to needs of a prescribed therapy. For example, in some embodiments, the tissue interface 108 may be a foam having pore sizes in a range of 400-600 microns. The tensile strength of the tissue interface 108 may also vary according to needs of a prescribed therapy. For example, the tensile strength of a foam may be increased for instillation of topical treatment solutions. In one non-limiting example, the tissue interface 108 may be an open-cell, reticulated polyurethane foam such as the foam employed in the V.A.C.® GRANUFOAM™ Dressing or the foam employed in the V.A.C. VERAFLO™ Dressing, both available from available from Acelity L.P., Inc. of San Antonio, Tex.

The tissue interface 108 may be either hydrophobic or hydrophilic. In an example in which the tissue interface 108 may be hydrophilic, the tissue interface 108 may also wick fluid away from a tissue site, while continuing to distribute negative pressure to the tissue site. The wicking properties of the tissue interface 108 may draw fluid away from a tissue site by capillary flow or other wicking mechanisms. An example of a hydrophilic foam is a polyvinyl alcohol, open-cell foam such as the foam employed in the V.A.C. WHITEFOAM™ Dressing available from Acelity L.P., Inc. of San Antonio, Tex. Other hydrophilic foams may include those made from polyether. Other foams that may exhibit hydrophilic characteristics include hydrophobic foams that have been treated or coated to provide hydrophilicity.

The tissue interface 108 may further promote granulation at a tissue site when pressure within the sealed therapeutic environment is reduced. For example, any or all of the surfaces of the tissue interface 108 may have an uneven, coarse, or jagged profile that can induce microstrains and stresses at a tissue site if negative pressure is applied through the tissue interface 108.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include without limitation polycarbonates, polyfumarates, and capralactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 106 may provide a bacterial barrier and protection from physical trauma. The cover 106 may also be constructed from a material that can reduce evaporative losses and provide a fluid seal between two components or two environments, such as between a therapeutic environment and a local external environment. The cover 106 may be, for example, an elastomeric film or membrane that can provide a seal adequate to maintain a negative pressure at a tissue site for a given negative-pressure source. The cover 106 may have a high moisture-vapor transmission rate (MVTR) in some applications. For example, the MVTR may be at least 300 g/m² per twenty-four hours in some embodiments. In some example embodiments, the cover 106 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained.

In some embodiments, the cover 106 may form a sealed space 107 at the tissue site. An attachment device may be used to attach the cover 106 to an attachment surface, such as undamaged epidermis, a gasket, or another cover. The attachment device may take many forms. For example, an attachment device may be a medically-acceptable, pressure-sensitive adhesive that extends about a periphery, a portion, or an entire sealing member. In some embodiments, for example, some or all of the cover 106 may be coated with an acrylic adhesive having a coating weight between 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

A controller, such as the controller 110, may be a microprocessor or computer programmed to operate one or more components of the therapy system 100, such as the negative-pressure source 104. In some embodiments, for example, the controller 110 may be a microcontroller, which generally comprises an integrated circuit containing a processor core and a memory programmed to directly or indirectly control one or more operating parameters of the therapy system 100. Operating parameters may include the power applied to the negative-pressure source 104, the pressure generated by the negative-pressure source 104, or the pressure distributed to the tissue interface 108, for example. The controller 110 is also preferably configured to receive one or more input signals, such as a feedback signal, and programmed to modify one or more operating parameters based on the input signals.

Sensors, such as the pressure sensor 120 or the electric sensor 122, are generally known in the art as any apparatus operable to detect or measure a physical phenomenon or property, and generally provide a signal indicative of the phenomenon or property that is detected or measured. For example, the pressure sensor 120 and the electric sensor 122 may be configured to measure one or more operating parameters of the therapy system 100. In some embodiments, the pressure sensor 120 may be a transducer configured to measure pressure in a pneumatic pathway and convert the measurement to a signal indicative of the pressure measured. In some embodiments, for example, the pressure sensor 120 may be a piezoresistive strain gauge. The electric sensor 122 may optionally measure operating parameters of the negative-pressure source 104, such as the voltage or current, in some embodiments. Preferably, the signals from the pressure sensor 120 and the electric sensor 122 are suitable as an input signal to the controller 110, but some signal conditioning may be appropriate in some embodiments. For example, the signal may need to be filtered or amplified before it can be processed by the controller 110. Typically, the signal is an electrical signal, but may be represented in other forms, such as an optical signal.

The therapy system 100 may include a fluid container, such as a vessel 112, coupled to the dressing 102 and to the negative-pressure source 104. For example, in some embodiments a tube may mechanically and fluidly couple the dressing 102 to the vessel 112 such that the negative-pressure source 104 may be indirectly coupled to the dressing 102 through the vessel 112. The vessel 112 may be configured to manage exudates and other fluids withdrawn from a tissue site.

### Vessel

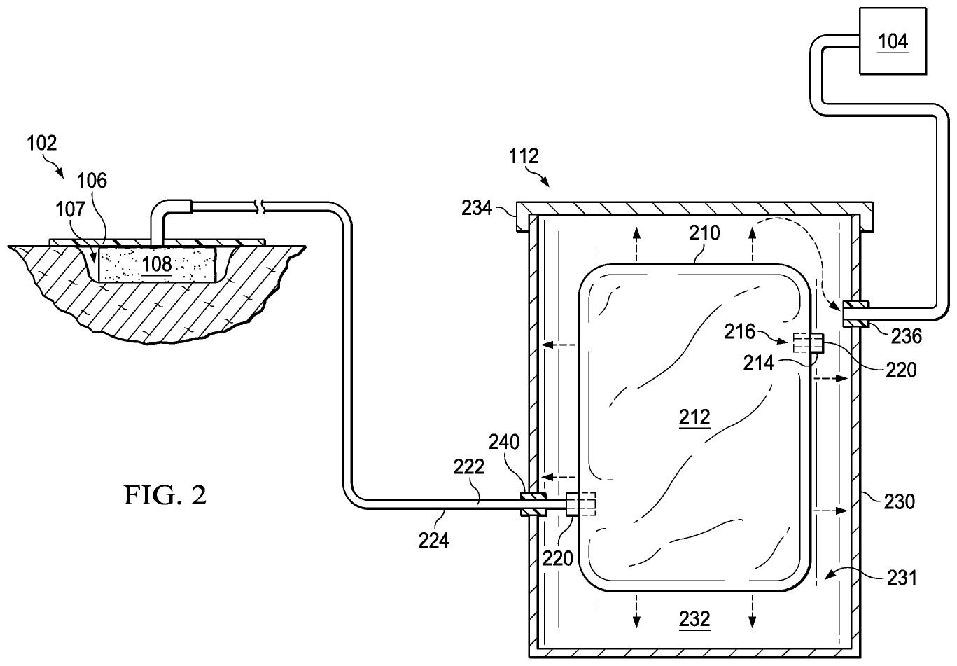

FIG. 2 illustrates a schematic view of an embodiment of a vessel 112 configured to manage exudates and other fluids withdrawn from a tissue site. In some embodiments, the vessel 112 may comprise a flexible container 210 and a canister 230. The flexible container 210 may be configured to be disposed within the canister 230.

### Flexible Container

In some embodiments, the flexible container 210 may be a bag, a pouch, a bellows-container, a bottle having one or more collapsible (e.g., accordion-like) walls, or combinations thereof. For example, the flexible container 210 may be generally characterized as collapsible and/or expandable. In some embodiments, the flexible container 210 may be configured to be collapsed such that the flexible container 210 occupies a relatively small amount of space, for example, for transport or storage. The flexible container 210 may be expanded such that the flexible container 210 will define an internal space 212 having a maximum internal volume when fully expanded. The flexible container 210 may have any suitable maximum internal volume, for example, from about 0.5 L to about 2.5 L, or from about 1.0 L to about 1.5 L. The flexible container 210 may have any suitable shape or orientation. For example, the flexible container 210 may be described as generally conical, tapered, pyramidal, or cubic.

In some embodiments, the flexible container 210 may be formed from any suitable material or assemblage of materials. The flexible container 210 may comprise a suitable film material, such as a plastic or a resin-based material, for example that may be formed into the flexible container 210. Examples of materials that may be used to form such the flexible container 210 may include, but are not limited to, films such as low-density linear polyethylene (LLDPE), low-density polyethylene (LDPE), high-density polyethylene (HDPE), polypropylene (PP), polyvinyl chloride (PVC), ethylene vinyl acetate (EVA), polyester, polyurethane (PU or PUR), or combinations thereof. In some embodiments, the flexible container 210 may be formed by joining two or more sheets of the material, for example, via a weld or adhesive.

In some embodiments, the flexible container 210 may include an absorbent material within the internal space 212, such as a super-absorbent polymer (SAP). In some embodiments, the absorbent material may swell, for example, so as to increase up to 1,000 times its original volume when fully hydrated with an aqueous fluid. The absorbent material may be in a dry form prior to contact with an aqueous fluid, for example, having a particulate form. Examples of absorbent materials may include a cross-linked homopolymer of acrylic acid or acrylate, acrylamide, ethylene, maleic anhydride, methacrylic acid, vinyl acetate, vinyl alcohol, acrylonitrile, hydroxyethylmethacrylate, carboxymethylcellulose, ethylene oxide, propylene oxide, vinylpyrrolidone, or styrenesulfonate; and copolymers of any of the foregoing monomers; and combinations thereof.

In some embodiments, the flexible container 210 may be configured to provide a route of pressure communication between the internal space 212 and an external space 231 outside the flexible container 210. Additionally, the flexible container 210 may also be configured such that the route of pressure communication between the internal space 212 and the external space 231 may be configured to restrict liquid communication between the internal space 212 and the external space 231. For example, the flexible container 210 may be configured to allow pressure to be communicated between the internal space 212 and the external space 231 while restricting liquid communication between the internal space 212 and the external space 231.

In some embodiments, the flexible container 210 may comprise a pressure port 214 defining a first flowpath 216. The first flowpath 216 may extend between the internal space 212 and the external space 231. The flexible container 210 may be configured to allow pressure to be communicated via the first flowpath 216 while restricting liquid communication via the first flowpath 216. For example, in some embodiments, the flexible container 210 may comprise a hydrophobic filter 218 configured to control fluid communication, including pressure communication and liquid communication, via the first flowpath 216.

In some embodiments, the hydrophobic filter 218 may be generally configured to restrict liquid communication from the internal space 212 to the external space while allowing gas communication. For example, the hydrophobic filter 218 may comprise a material that is generally liquid impermeable and vapor permeable. The hydrophobic filter 218 may be, for example, a porous, sintered polymer. As an example, the hydrophobic filter 218 may comprise a material manufactured under the designation MMT-314 by W.L. Gore & Associates, Inc. of Newark, Del., or a similar material.

In some embodiments, the hydrophobic filter 218 may be configured to interact with the pressure port 214, for example, so as to substantially preclude liquid from passing through the pressure port 214 via the first flowpath 216. For example, in some embodiments the hydrophobic filter 218 may be sized to fit within or over the dimensions of the first flowpath 216. In some embodiments, the hydrophobic filter 218 may be in the form of a membrane or a layer. The hydrophobic filter 218 may be disposed within and/or over the pressure port 214. For example, in various embodiments, the hydrophobic filter 218 may be held in place with respect to the pressure port 214 via a suitable interface such as an adhesive or a mechanical interface such as a threaded interface.

In some embodiments, the flexible container 210 may comprise a plurality of pressure ports substantially similar to the pressure port 214, each configured allow pressure to be communicated between the internal space 212 and the external space 231 while restricting liquid communication between the internal space 212 and the external space 231. The plurality of pressure ports 214, for example, each defining a flowpath and having a hydrophobic filter 218 configured to restrict liquid communication and to allow gas communication via the respective flowpaths. In various embodiments, the flexible container 210 may comprise two, three, four, five, six, seven, eight, nine, ten, or more ports, configured as the pressure port 214.

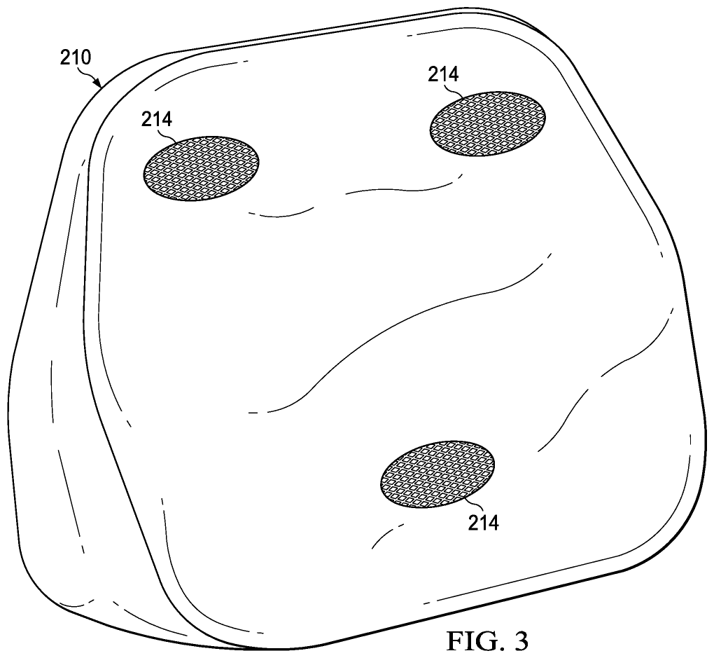

In some embodiments, any two or more ports, for example, any two or more of a first port, a second port, and a third port, may be located on generally opposing surfaces of the flexible container 210 and/or at generally opposite sides of the flexible container 210. FIG. 3 illustrates an embodiment of the flexible container 210 having a plurality of pressure ports 214, each being configured to allow pressure communication between the internal space 212 and the external space 231 while restricting liquid communication between the internal space 212 and the external space 231. Generally, the first port, the second port, and the third port are disposed at different positions about the flexible container 210. In some embodiments, positioning the pressure ports 214 on generally opposing surfaces of the flexible container 210 and/or at generally opposite sides of the flexible container 210 may help to ensure that, in the event that a given port may become occluded such as by fluid retained within the internal space or otherwise unable to communicate pressure between the internal space 212 and the external space 231, one or more other pressure ports 214 may remain capable of pressure communication.

In some embodiments, the one or more pressure ports 214 may be configured to communicate pressure at a desired rate. In some embodiments, the flexible container 210 may be configured for use with a particular negative-pressure source, for example, a negative-pressure source capable of generating negative pressure at a particular rate. In some embodiments, the one or more pressure ports 214 may be configured to cumulatively communicate pressure between the internal space 212 and the external space 231 at a rate that is about equal to the rate at which a negative-pressure source connected thereto may generate negative pressure, or at a rate that is at greater than the rate at which a negative-pressure source connected thereto may generate negative pressure. For example, the one or more pressure ports 214 may be configured to cumulatively communicate pressure between the internal space 212 and the external space 231 at a rate that is at least about 110%, or about 120%, or about 130%, or about 140%, or about 150%, or about 160%, or about 170%, or about 180%, or about 190%, or about 200%, or about 225%, or about 250% of the rate at which the negative-pressure source with which the flexible container 210 is used is configured to generate negative pressure.

In some embodiments, the flexible container 210 may be configured to provide a route of fluid communication between the internal space 212 and the sealed space 107. Referring again to FIG. 2, in some embodiments the flexible container 210 may comprise a fluid port 220 defining at least a portion of a second flowpath 222. The second flowpath 222 may extend between the internal space 212 and the sealed space 107.

In some embodiments, the fluid port 220 may be fluidly coupled to the sealed space 107 via a tube 224 or other fluid conductor integral with the fluid port 220 and defining at least a portion of the second flowpath 222. Additionally or alternatively, in some embodiments the fluid port 220 may comprise a suitable fitting or coupler, for example, to provide for connection to a fluid conduit. Examples of such fittings and couplers may include, but are not limited to, push-to-connect fittings, compression fittings, barb fittings, and the like.

### Canister

The canister 230 may be rigid and define a chamber 232 having a fixed volume. For example, the chamber 232 may define an internal volume from about 0.5 L to about 2.5 L, or from about 1.0 to about 1.5 L. In some embodiments, the canister 230 may include sidewalls, a base, and a lid 234 cooperatively defining the chamber 232. In various embodiments, the canister 230 may have any suitable shape, design, and orientation. In some embodiments, for example, the canister 230 may be described as generally conical, tapered, pyramidal, or cubic. Also, the canister 230 may be described as having a cross-section in a horizontal plane that is circular, oval, square, rectangular, triangular, pentagonal, hexagonal, or any other suitable shape.

The canister 230 may be generally adapted to be substantially fluid-tight, for example, such that a negative pressure applied to the chamber 232 may be retained with little dissipation of the negative pressure. For example, in some embodiments, the engagement between the lid 234 and the canister 230 may include a suitable seal, examples of which include but are not limited to, an O-ring, a T-seal, a gasket, and a compression seal, as suitable. The lid 234 may be removable from the canister 230 or may be hinged with respect to the canister 230.

In some embodiments, the canister 230 may be configured to provide a route of fluid communication between the internal space 212 and the negative-pressure source 104. For example, in some embodiments the canister 230 may include a connection port 236. The connection port 236 may include a suitable fitting or coupler, for example, to provide for connection to a fluid conduit. Examples of such fittings and couplers may include, but are not limited to, push-to-connect fittings, compression fittings, barb fittings, and the like. In some embodiments, the connection port 236 may comprise a filter, such as the hydrophobic filter disclosed herein. For example, the filter may ensure that liquids, such as wound exudate, are not drawn into the negative-pressure source 104 in the event of leakage from the flexible container 210.

In some embodiments, the canister 230 may be configured to provide one or more routes of fluid communication from the connection port 236 throughout the chamber 232. In some embodiments, the flow channels may be effective to ensure pressure communication between the connection port 236 and the fluid ports 220 of the flexible container 210 when the flexible container 210 is disposed within the chamber 232. For example, in some embodiments one or more interior surfaces of the canister 230 may include one or more flow channels.

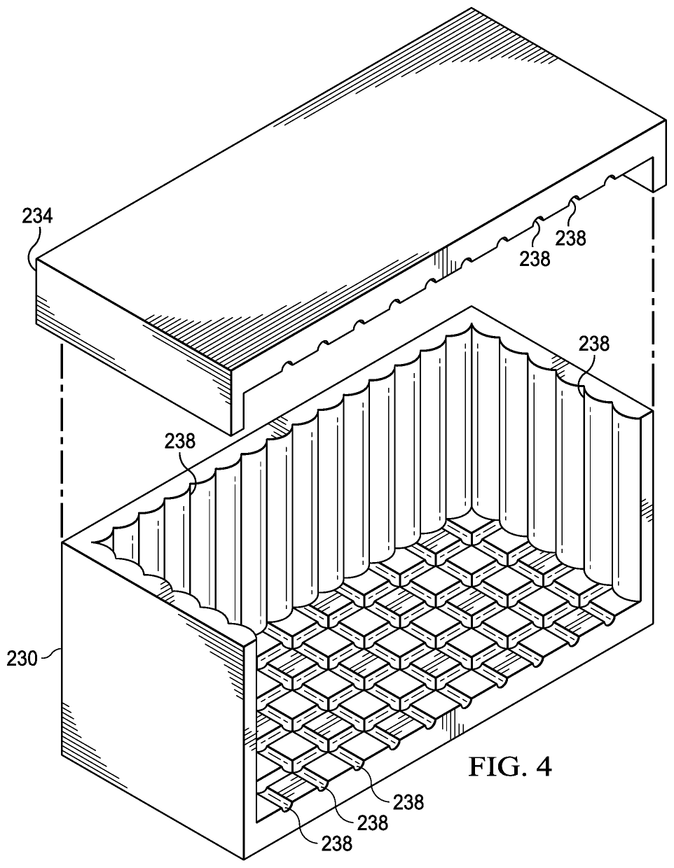

In some embodiments, the flow channels may extend across one or more interior surfaces of the canister 230 in a suitable pattern, for example, radially, longitudinally, or in the form of a grid. The flow channels may be formed by ridges, ribs, grooves, depressions, or combinations thereof. For example, FIG. 4 is a cross-sectional view of an embodiment of the canister 230 having flow channels 238. For example, the flow channels 238 within the base of the canister 230 and the within the lid 234 may be configured in a grid pattern and the flow channels 238 within the sidewalls of the canister 230 may be vertically-oriented, longitudinal channels.

In some embodiments, the canister 230 may be configured to provide a passageway for a fluid conduit extending between the flexible container 210 and the dressing 102. For example, referring again to FIG. 2, the canister 230 may comprise a passageway 240 configured to receive the tube 224 providing fluid communication between the internal space 212 of the flexible container 210 and the sealed space 107. In some embodiments, the passageway 240 may sealingly engage the tube 224 such that the chamber 232 remains substantially fluid-tight. The passageway 240 may be disposed at any suitable location on the canister, for example, within a sidewall or within the lid 234. In some embodiments, the passageway 240 may be comprise a slot within each of a sidewall of the canister 230 and the lid 234 that, when aligned, form the passageway 240.

In some embodiments, the canister 230 may be configured to provide a secondary route of fluid communication between the sealed space 107 and the negative-pressure source 104. For example, in some embodiments the canister 230 may include a portion of the secondary route of fluid communication between the sealed space 107 and the negative-pressure source 104. In various embodiments, such a secondary route of fluid communication between the sealed space 107 and the negative-pressure source 104 may be effective for obtaining data regarding one or more parameters or conditions at the sealed space 107 or as an alternative route to communicate negative pressure from the negative-pressure source 104 to the sealed space. For example, in some embodiments the secondary route of fluid communication may be used to monitor the pressure within the sealed space 107.

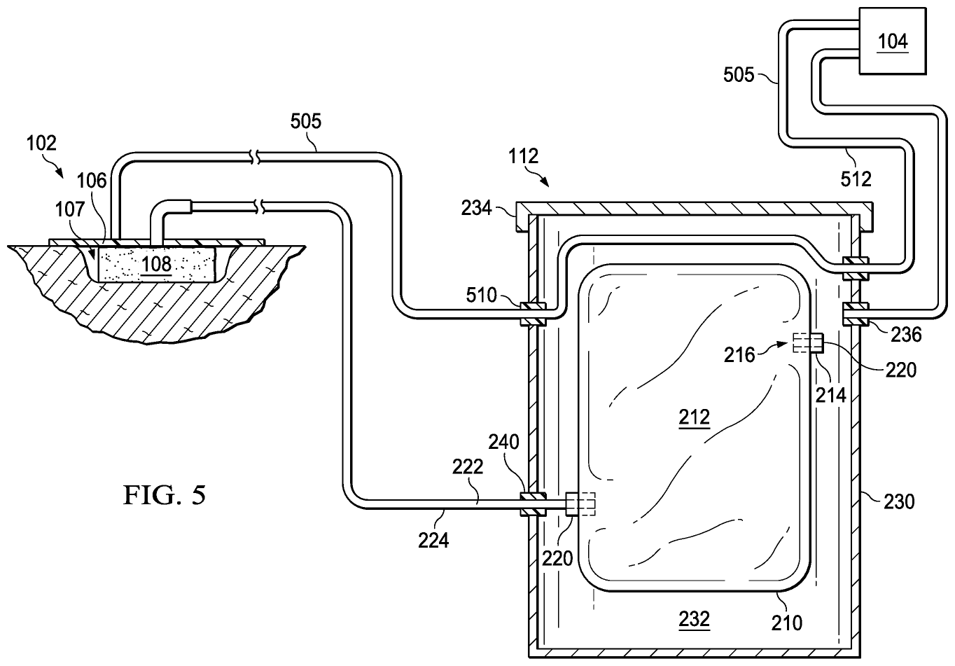

For example, FIG. 5 illustrates a schematic view of an embodiment of a vessel 112 configured to provide a secondary route of fluid communication 505 between the negative-pressure source 104 and the sealed space 107. In the embodiment of FIG. 5, the secondary route of fluid communication 505 includes a portion extending between the sealed space 107 and the vessel 112, a portion extending through or around the vessel 112, and a portion extending from the vessel 112 to the negative-pressure source 104. In some embodiments, the tube 224 may further include the secondary route of fluid communication 505. For example, the tube 224 may be a multi-lumen conduit configured to provide multiple, separate routes of fluid communication between various components. Examples of such multi-lumen conduits may include those suitably used in conjunction with a dressing interface such as the SENSAT.R.A.C.™ Dressing available from Acelity L.P. of San Antonio, Tex.

In some embodiments, the vessel 112 may further comprise a first auxiliary port 510 and a second auxiliary port 512. The first auxiliary port 510 and second auxiliary port 512 may provide for fluid connection between the secondary route of fluid communication 505 and the vessel 112. In embodiments where a multi-lumen conduit is used, the first auxiliary port 510 may be incorporated with the passageway 240 and the second auxiliary port 512 may be incorporated with the connection port 236, for example, so as to enable connection to the multi-lumen conduit. In some embodiments, the first auxiliary port 510 and/or the second auxiliary port 512 may comprise a filter, such as the hydrophobic filter disclosed herein.

In various embodiments, the portion of the secondary route of fluid communication 505 extending through or around the vessel 112 may take any suitable pathway between the first auxiliary port 510 and the second auxiliary port 512. For example, the vessel 112 may include a conduit that provides fluid connection between the first auxiliary port 510 and the second auxiliary port 512. In some embodiments, such a conduit may extend through the chamber 232 (e.g., with making fluid connection to the chamber 232). Additionally or alternatively, in some embodiments, the conduit may be incorporated within the structure of the vessel 112 (e.g., within the walls of the canister 230).

Alternatively, in some embodiments the vessel 112 may be used in a system having a secondary route of fluid communication between the sealed space 107 and the negative-pressure source 104, but where the vessel does not include a portion of the secondary route of fluid communication. For example, in some embodiments a secondary route of fluid communication may extend between the sealed space 107 and the negative-pressure source 104 without fluid or mechanical connection to the vessel 112 (e.g., around the vessel 112).

Methods

The vessel 112 may be employed in the context of a negative-pressure therapy, for example, to collect wound liquids, such as blood, water, and wound exudate, removed from a tissue site.

For example, in a therapy method, the tissue interface 108 may be placed within, over, on, or otherwise proximate to a tissue site. The cover 106 may be placed over the tissue interface 108 and sealed to an attachment surface near the tissue site, for example, to form the sealed space 107. For example, the cover 106 may be sealed to undamaged epidermis peripheral to a tissue site. Thus, the dressing 102 can provide a sealed therapeutic environment proximate to a tissue site, substantially isolated from the external environment.

The vessel 112 may also be prepared for use in the therapy method. For example, the flexible container 210 may be disposed within the chamber 232 of the canister 230. Suitable fluid conduits may be connected to fluidly couple the internal space 212 of the flexible container 210 to the sealed space 107 and to fluidly couple the chamber 232 to the negative-pressure source 104. The canister 230 may be closed and sealed, for example, such that the chamber 232 is substantially fluid-tight.

The negative-pressure source 104 may supply negative pressure to reduce the pressure within the sealed space 107, for example, at the dressing 102. For example, in operation, a negative pressure may be applied to the chamber 232 via the operation of the negative-pressure source 104. The application of the negative pressure to the chamber 232 may cause a negative pressure to be communicated via one or more pressure ports 214 in the flexible container 210 to the internal space 212 of the flexible container and from the internal space 212 to the sealed space 107.

In some embodiments, the application of negative pressure to the sealed space 107 may be effective to withdraw or remove wound liquids from the tissue site. As the wound liquids are withdrawn from the tissue site, the liquids may be collected within the internal space 212 of the flexible container 210. The liquids may be retained within the internal space 212 while negative pressure continues to be applied via the pressure ports 214. For example, the hydrophobic filters 218 may allow pressure to be communicated between the internal space 212 of the flexible container 210 and the chamber 232 while the flexible container 210 is disposed within the chamber 232 and, at the same time restrict liquid communication between the internal space 212 and the chamber 232.

In some embodiments, wound fluids may be drawn into and retained within the internal space 212 of the flexible container 210 until the therapy is concluded or the flexible container 210 is substantially or entirely filled. The flexible container 210 may be removed from the canister 230, along with the wound fluids retained therein, and disposed of. The canister 230 may be sterilized and reused in additional therapies.

Advantages

In various embodiments, a therapy system like therapy system 100 or components thereof, such as the vessel 112, may be advantageously employed in the provision of negative pressure therapy to a patient. For example, because the wound fluids are retained within the flexible container 210, the canister 230 may be used in multiple therapies, with limited risk of becoming contaminated. As such, the vessel 112 may decrease the costs and overhead associated with the provision of negative-pressure therapy. For example, because the canister 230 may be reused while only the flexible container 210 may be disposed of, the number of the canisters 230 that must be housed at healthcare facilities can be dramatically decreased. Instead, healthcare facilities need only retain substantial numbers of the flexible containers 210, which may be relatively less costly and require relatively less shelf-space.

While shown in a few illustrative embodiments, a person having ordinary skill in the art will recognize that the systems, apparatuses, and methods described herein are susceptible to various changes and modifications. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 102, the vessel 112, or both may be eliminated or separated from other components for manufacture or sale. In other example configurations, the controller 110 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

What is claimed is:

1. A system for treating a tissue site with reduced pressure, the system comprising:
a distribution component;
a negative-pressure source; and
a fluid-collection vessel comprising:
a rigid canister defining a chamber; and
a flexible container configured to receive a fluid from the distribution component and to provide a route of pressure communication while restricting liquid communication between an internal volume of the flexible container and the chamber of the rigid canister when the flexible container is disposed within the chamber of the rigid canister.

2. The system of claim 1, wherein the distribution component comprises a dressing disposed at the tissue site.

3. The system of one of claims 1-2, wherein the flexible container comprises a first port configured to provide the route of pressure communication between the internal volume of the flexible container and the chamber of the rigid canister.

4. The system of claim 3, wherein the first port is configured to restrict liquid communication between the internal volume of the flexible container and the chamber of the rigid canister.

5. The system of one of claims 3-4, wherein the first port comprises a hydrophobic filter.

6. The system of claim 5, wherein the hydrophobic filter is configured to allow pressure communication and to restrict liquid communication.

7. The system of one of claims 3-6, wherein the flexible container comprises a second port configured to provide the route of pressure communication between the internal volume of the flexible container and the chamber of the rigid canister.

8. The system of claim 7, wherein the first port is disposed on a first surface of the flexible container and the second port is disposed on a second surface of the flexible container.

9. The system of one of claims 1-8, wherein the rigid canister is configured to provide a route of fluid communication to the negative-pressure source.

10. The system of one of claims 1-9, wherein the rigid canister is configured to provide a route of fluid communication between the distribution component and the flexible container.

11. The system of one of claim 1-10, wherein an interior surface of the rigid canister comprises a plurality of flow channels.

12. The system of claim 11, wherein the plurality of flow channels is configured to provide a portion of a route of pressure communication between the negative-pressure source and the route of pressure communication between an internal volume of the flexible container and the chamber of the rigid canister.

13. The system of one of claims 11-12, wherein the plurality of flow channels is formed from a plurality of ridges or ribs.

14. The system of one of claims 1-13, further comprising a route of pressure communication between the distribution component and the negative-pressure source, wherein the route of pressure communication between the distribution component and the negative-pressure source includes the chamber of the rigid canister.

15. A vessel for collecting liquid from a tissue site being treated with a reduced pressure, the vessel comprising:
a rigid canister defining a chamber; and
a flexible container configured to receive a fluid from the distribution component and to provide a route of pressure communication while restricting liquid communication between an internal volume of the flexible container and the chamber of the rigid canister when the flexible container is disposed within the chamber of the rigid canister.

16. The vessel of claim 15, wherein the distribution component comprises a dressing disposed at the tissue site.

17. The vessel of one of claims 15-16, wherein the flexible container comprises a first port configured to provide the route of pressure communication between the internal volume of the flexible container and the chamber of the rigid canister.

18. The vessel of claim 17, wherein the first port is configured to restrict liquid communication between the internal volume of the flexible container and the chamber of the rigid canister.

19. The vessel of one of claims 17-18, wherein the first port comprises a hydrophobic filter.

20. The vessel of claim 19, wherein the hydrophobic filter is configured to allow pressure communication and to restrict liquid communication.

21. The vessel of one of claims 17-20, wherein the flexible container comprises a second port configured to provide the route of pressure communication between the internal volume of the flexible container and the chamber of the rigid canister.

22. The vessel of claim 21, wherein the first port is disposed on a first surface of the flexible container and the second port is disposed on a second surface of the flexible container.

23. The vessel of one of claims 15-22, wherein the rigid canister is configured to provide a route of fluid communication to a negative-pressure source.

24. The vessel of one of claims 15-23, wherein the rigid canister is configured to provide a route of fluid communication between the distribution component and the flexible container.

25. The vessel of one of claim 15-24, wherein an interior surface of the rigid canister comprises a plurality of flow channels.

26. The vessel of claim 25, wherein the plurality of flow channels is configured to provide a portion of a route of pressure communication between the negative-pressure source and the route of pressure communication between an internal volume of the flexible container and the chamber of the rigid canister.

27. The vessel of one of claims 25-26, wherein the plurality of flow channels is formed from a plurality of ridges or ribs.

28. The vessel of one of claims 15-27, further comprising a route of pressure communication between the distribution component and the negative-pressure source, wherein the route of pressure communication between the distribution component and the negative-pressure source includes the chamber of the rigid canister.

29. A method for treating a tissue site with reduced pressure, the method comprising:
sealing the tissue site at a distribution component;
fluidly coupling a negative-pressure source to the distribution component;
drawing fluid from the distribution component with the negative-pressure source; and
collecting at least a portion of the fluid in a vessel comprising:
a rigid canister defining a chamber; and
a flexible container configured to receive a fluid from the distribution component and to provide a route of pressure communication and to restrict liquid communication between an internal volume of the flexible container and the chamber of the rigid canister when the flexible container is disposed within the chamber of the rigid canister.

30. The method of claim 29, wherein the distribution component comprises a dressing disposed at the tissue site.

31. The method of one of claims 29-30, wherein the flexible container comprises a first port configured to provide the route of pressure communication between the internal volume of the flexible container and the chamber of the rigid canister.

32. The method of claim 31, wherein each of the first port is configured to restrict liquid communication between the internal volume of the flexible container and the chamber of the rigid canister.

33. The method of one of claims 31-32, wherein the first port comprises a hydrophobic filter.

34. The method of claim 33, wherein the hydrophobic filter is configured to allow pressure communication and to restrict liquid communication.

35. The method of one of claims 31-34, wherein the flexible container comprises a second port configured to provide the route of pressure communication between the internal volume of the flexible container and the chamber of the rigid canister.

36. The method of claim 35, wherein the first port is disposed on a first surface of the flexible container and the second port is disposed on a second surface of the flexible container.

37. The method of one of claims 29-36, wherein the rigid canister is configured to provide a route of fluid communication to the negative-pressure source.

38. The method of one of claims 29-37, wherein the rigid canister is configured to provide a route of fluid communication between the distribution component and the flexible container.

39. The method of one of claim 29-38, wherein an interior surface of the rigid canister comprises a plurality of flow channels.

40. The method of claim 39, wherein the plurality of flow channels is configured to provide a portion of a route of pressure communication between the negative-pressure source and the route of pressure communication between an internal volume of the flexible container and the chamber of the rigid canister.

41. The method of one of claims 39-40, wherein the plurality of flow channels are formed from a plurality of ridges or ribs.

42. The method of one of claims 29-41, further comprising sensing pressure within the distribution component via a route of pressure communication between the distribution component and the negative-pressure source, wherein the route of pressure communication between the distribution component and the negative-pressure source includes the chamber of the rigid canister.

43. A vessel for collecting fluids from a tissue site being treated reduced pressure treatment, the vessel comprising:
a rigid canister defining a chamber;
a first port in fluid communication with the chamber and adapted to be fluidly coupled to a negative-pressure source;
a second port extending into the chamber and adapted to be fluidly coupled to the tissue site for collecting fluids from the tissue site; and
a flexible container disposed within the chamber and forming an external space between the chamber and the flexible container, the flexible container having an inlet fluidly coupled to the second port for receiving fluids from the tissue site, an outlet in fluid communication with the external space, and a filter disposed within the outlet adapted to restrict communication of liquids from the flexible container into the external space, and wherein the first port is also in fluid communication with the external space.

44. The system of claim 43, wherein the filter comprises a hydrophobic filter.

45. The system of claim 44, wherein the hydrophobic filter is configured to allow pressure communication and to restrict liquid communication.

46. The system of one of claims 43-45, wherein the flexible container comprises a second outlet in fluid communication with the external space and a second filter disposed within the second outlet adapted to restrict communication of liquids from the flexible container into the external space.

47. The system of claim 46, wherein the outlet is disposed on a first surface of the flexible container and the second outlet is disposed on a second surface of the flexible container.

48. The system of one of claim 43-47, wherein an interior surface of the rigid canister comprises a plurality of flow channels.

49. The system of claim 48, wherein the plurality of flow channels is configured to provide a portion of a route of pressure communication between the negative-pressure source and the route of pressure communication between an internal volume of the flexible container and the chamber of the rigid canister.

50. The system of one of claims 48-49, wherein the plurality of flow channels is formed from a plurality of ridges or ribs.
